(19) United States
(12) Patent Application Publication
(10) Pub. No.: US 2022/0142475 A1
(43) Pub. Date: May 12, 2022

(54) OPHTHALMIC APPARATUS, AND RECORDING MEDIUM STORING OPHTHALMIC APPARATUS CONTROLLING PROGRAM

(71) Applicant: NIDEK CO., LTD., Gamagori-shi (JP)

(72) Inventors: Shinichi HASHIMOTO, Gamagori-shi (JP); Tomofumi FURUKAWA, Gamagori-shi (JP)

(73) Assignee: NIDEK CO., LTD., Gamagori-shi (JP)

(21) Appl. No.: 17/517,187

(22) Filed: Nov. 2, 2021

(30) Foreign Application Priority Data
Nov. 9, 2020 (JP) 2020-186795

(51) Int. Cl. A61B 3/15; A61B 3/00; G16H 30/40

(52) U.S. Cl. CPC A61B 3/152; G16H 30/40; A61B 3/0008

(57) ABSTRACT

An ophthalmic apparatus that examines an examinee's eye while an examination axis coincides with the examinee's eye, the ophthalmic apparatus including: a housing: an examination-purpose protrusion protruding along the examination axis toward the examinee's eye from an examinee's eye facing surface being a surface of the housing that faces the examinee's eye; an anterior segment imaging optical system configured to capture an image of the anterior segment of the examinee's eye; and a processor, wherein the processor executes: an anterior segment image acquisition step of acquiring the anterior segment image captured by the anterior segment imaging optical system; and a pupil position detection step of processing the acquired anterior segment image and detecting the position of the pupil of the examinee's eye while the influence of the shadow of the examination-purpose protrusion that appears in the anterior segment image is removed.

### CROSS-REFERENCE TO RELATED APPLICATION

This application claims priority from Japanese Patent Application No. 2020-186795 filed with the Japan Patent Office on Nov. 9, 2020, the entire content of which is hereby incorporated by reference.

### BACKGROUND

### 1. Technical Field

The present disclosure relates to an ophthalmic apparatus for examining an examinee's eye, and a recording medium including an ophthalmic apparatus control program for controlling the ophthalmic apparatus recorded thereon.

### 2. Related Art

A technology for detecting the position of the anterior segment of an examinee's eye is used, for example, when the position of an ophthalmic apparatus relative to the examinee's eye is adjusted to an appropriate relative position (in other words, when the ophthalmic apparatus is aligned relative to the examinee's eye). For example, an ophthalmic apparatus described in JP-A-2019-63043 detects edges in an image of the anterior segment of the examinee's eye, and detects the position of the pupil of the examinee's eye on the basis of, for example, the shape of the detected edges.

### SUMMARY

An ophthalmic apparatus according to the embodiment of the present disclosure examines an examinee's eye while an examination axis coincides with the examinee's eye, the ophthalmic apparatus including: a housing: an examination-purpose protrusion protruding along the examination axis toward the examinee's eye from an examinee's eye facing surface being a surface of the housing that faces the examinee's eye; an anterior segment imaging optical system configured to capture an image of the anterior segment of the examinee's eye; and a processor, wherein the processor executes: an anterior segment image acquisition step of acquiring the anterior segment image captured by the anterior segment imaging optical system; and a pupil position detection step of processing the acquired anterior segment image and detecting the position of the pupil of the examinee's eye while the influence of the shadow of the examination-purpose protrusion that appears in the anterior segment image is removed.

### BRIEF DESCRIPTION OF THE DRAWINGS

FIG. 1 is a left side view illustrating an external configuration of an ophthalmic apparatus 1;

FIG. 2 is a diagram illustrating an internal configuration of the ophthalmic apparatus 1;

FIG. 3 is a diagram illustrating optical systems of the ophthalmic apparatus 1;

FIG. 4 is a diagram illustrating an image of the skin of an examinee captured by an anterior segment imaging optical system 35;

FIG. 5 is a diagram illustrating an image that was captured by the anterior segment imaging optical system 35 while the pupil center was located below an examination axis IO;

FIG. 6 is a diagram illustrating an image that was captured by the anterior segment imaging optical system 35 while the examination axis 10 coincided with the pupil center and the corneal apex;

FIG. 7 is a flowchart of an automatic alignment process that is executed by the ophthalmic apparatus 1;

FIG. 8 is an explanatory diagram for explaining an example of a state where a pre-search area 91 is set in an anterior segment image 90; and

FIG. 9 is an explanatory diagram for explaining an example of a state where a pupil search area 92 is set in the anterior segment image 90.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In a known technology for detecting the position of the pupil of an examinee's eye on the basis of an anterior segment image captured, the accuracy of the detection of the position of the pupil may be influenced by, for example, an environment of photographing the anterior segment by an anterior segment imaging optical system. Therefore, a technology for allowing the detection of the position of the pupil of an examinee's eye with high accuracy regardless of, for example, the anterior segment photographing environment is being desired.

A typical object of the present disclosure is to provide an ophthalmic apparatus that can detect the position of the pupil of an examinee's eye with high accuracy and a recording medium including an ophthalmic apparatus control program recorded thereon.

An ophthalmic apparatus provided by the typical embodiment according to the embodiment examines an examinee's eye while an examination axis coincides with the examinee's eye, the ophthalmic apparatus including: a housing: an examination-purpose protrusion protruding along the examination axis toward the examinee's eye from an examinee's eye facing surface being a surface of the housing that faces the examinee's eye; an anterior segment imaging optical system configured to capture an image of the anterior segment of the examinee's eye; and a processor, wherein the processor executes: an anterior segment image acquisition step of acquiring the anterior segment image captured by the anterior segment imaging optical system; and a pupil position detection step of processing the acquired anterior segment image and detecting the position of the pupil of the examinee's eye while the influence of the shadow of the examination-purpose protrusion that appears in the anterior segment image is removed.

A recording medium where an ophthalmic apparatus control program to be executed by an ophthalmic apparatus provided by the typical embodiment according to the embodiment examines an examinee's eye while an examination axis coincides with the examinee's eye is recorded, wherein the ophthalmic apparatus includes: a housing: an examination-purpose protrusion protruding along the examination axis toward the examinee's eye from an examinee's eye facing surface being a surface of the housing that faces the examinee's eye; an anterior segment imaging optical system configured to capture an image of the anterior segment of the examinee's eye; and a processor, and the ophthalmic apparatus control program is executed by the processor of the ophthalmic apparatus to cause the ophthalmic apparatus to execute: an anterior segment image acquisition step of acquiring the anterior segment image captured by the anterior segment imaging optical system; and a pupil position detection step of processing the acquired anterior segment image and detecting the position of the pupil of the examinee's eye while the influence of the shadow of the examination-purpose protrusion that appears in the anterior segment image is removed.

In the ophthalmic apparatus and the recording medium including the ophthalmic apparatus control program recorded thereon according to the present disclosure, the position of the pupil of an examinee's eye is detected with high accuracy.

### <Overview>

The ophthalmic apparatus illustrated by example in the present disclosure examines an examinee's eye while an examination axis coincides with the examinee's eye. The ophthalmic apparatus according to the present disclosure includes a housing, an examination-purpose protrusion, an anterior segment imaging optical system, and a processor. The examination-purpose protrusion protrudes along the examination axis toward the examinee's eye from an examinee's eye facing surface being a surface of the housing that faces the examinee's eye. The anterior segment imaging optical system captures an anterior segment image of the examinee's eye. The processor is responsible for processing control of the ophthalmic apparatus. The processor executes an anterior segment image acquisition step and a pupil position detection step. In the anterior segment image acquisition step, the processor acquires the anterior segment image captured by the anterior segment imaging optical system. In the pupil position detection step, the processor processes the acquired anterior segment image and detects the position of the pupil of the examinee's eye while the influence of the shadow of the examination-purpose protrusion that appears in the anterior segment image is removed.

The examination-purpose protrusion (for example, a nozzle through which fluid that is blown on the cornea of the examinee's eye passes) provided to the housing of the ophthalmic apparatus protrudes toward the examinee's eye along the examination axis, and the examination axis coincides with the examinee's eye during the examination. Therefore, in terms of the structure, it is difficult to prevent the shadow of the examination-purpose protrusion protruding toward the examinee's eye from appearing in the anterior segment image captured by the anterior segment imaging optical system. The pupil of the examinee's eye appears darker than tissues around the pupil (for example, the iris, the sclera, and the eyelid) in an anterior segment image where the shadow of the examination-purpose protrusion does not appear. However, the brightness of the tissues around the pupil that appear brighter than the pupil under normal circumstances may decrease due to the influence of the shadow of the examination-purpose protrusion in an anterior segment image where the shadow of the examination-purpose protrusion appears. As a result, the boundary between the tissues near the pupil (for example, the boundary between the pupil and the iris) becomes unclear. Therefore, it is difficult to maintain high detection accuracy in the known pupil detection method that uses edge detection.

In contrast, the ophthalmic apparatus according to the present disclosure processes an anterior segment image to detect the position of the pupil of an examinee's eye while the influence of the shadow of the examination-purpose protrusion that appears in the anterior segment image is removed. Therefore, the position of the pupil of the examinee's eye is detected with high accuracy regardless of the presence or absence of the shadow of the examination-purpose protrusion appearing in an anterior segment image, the shadow being difficult to avoid its appearance in terms of the structure.

The method for using a result of the detection of the position of the pupil can be selected as appropriate. A result of the detection of the position of the pupil (for example, the position of the pupil center) may be used, for example, when the position of the ophthalmic apparatus relative to the examinee's eye is adjusted to an appropriate relative position (in other words, when the ophthalmic apparatus is aligned relative to the examinee's eye). In this case, the ophthalmic apparatus can be aligned even if a raster is not formed on the cornea in contrast to a case of being aligned on the basis of a raster formed on the cornea. After an alignment is performed on the basis of the position of the pupil, an alignment may be performed on the basis of the raster formed on the cornea to improve the final alignment accuracy.

In the pupil position detection step, the processor may execute a pupil search area setting step, a dark region's area acquisition step, and an area detection step. In the pupil search area setting step, the processor sets a pupil search area of a predetermined shape in a part of the image area of the anterior segment image. In the dark region's area acquisition step, the processor acquires the area of a dark region being a region having a luminance value equal to or less than a threshold in the pupil search area. In the area detection step, the processor detects the position of the pupil of the examinee's eye on the basis of the acquired area of the dark region.

In many cases, the luminance of a portion of the tissues around the pupil that appear in the anterior segment image, a portion where the shadow of the examination-purpose protrusion overlaps, is lower than the luminance of a portion where the shadow does not overlap, but is higher than the luminance of the pupil portion. Therefore, the dark region having a luminance value equal to or less than the threshold is highly likely to be the pupil region that appears darker than a shadowed tissue around the pupil that appears dark. Hence, the position of the pupil is detected on the basis of the area of the dark region to detect the position of the pupil of the examinee's eye with high accuracy regardless of the presence or absence of the shadow that appears in the anterior segment image.

In other words, the ophthalmic apparatus illustrated by example in the present disclosure easily detects the position of the pupil with high accuracy even if the boundary between the tissues near the pupil is unclear due to the shadow that appears on the tissues around the pupil. Moreover, a method is also conceivable which judges that the pupil is located in the pupil search area if a total or average luminance value of the pupil search area is low. However, a low total or average luminance value of the area results from a general reduction in brightness in the area due to the shadow and from the inclusion of the pupil that appears dark in a part of the area. Therefore, it is difficult to ensure high detection accuracy in the method that uses a total or average luminance value of the area. In contrast, the ophthalmic apparatus illustrated by example in the present disclosure handles the dark region having a luminance value equal to or less than the threshold as the pupil region and therefore has a reduced possibility of false detection of a pupil neighboring region where the shadow overlaps, as the pupil region.

The processor may execute a threshold setting step of setting a threshold for comparing a luminance value. An appropriate threshold for judging whether or not each pixel is a pixel in the pupil region varies depending on, for example, the brightness of a location where the ophthalmic apparatus is installed, or the race of the examinee. Therefore, the threshold is set as appropriate to further improve the accuracy to detect the position of the pupil.

A specific method for setting the threshold can be selected as appropriate. For example, the processor may set the threshold in response to an instruction inputted by a user. Moreover, a peak of the luminance value in the pupil region, and a peak of the luminance value of the pupil neighboring region where the shadow overlaps tend to appear in a histogram of the luminance values of the pixels in the anterior segment image. Therefore, the processor may detect a luminance value between the peak luminance value of the pupil region and the peak luminance value of the pupil neighboring region where the shadow overlaps, from a histogram of the anterior segment image, and set the detected luminance value as the threshold.

A specific method for detecting the position of the pupil on the basis of the area of the dark region can also be selected as appropriate. For example, the processor may detect a pupil search area including a dark region whose area to be acquired is the largest, as an area where the pupil is located. Moreover, if a plurality of pupil search areas including a dark region whose area is equal to or greater than a threshold is detected, the processor may detect, as the area where the pupil is located, a pupil search area including a dark region whose area is the closest to a general area of the pupil (for example, the average of the areas of the pupils of ordinary people) among the plurality of pupil search areas detected.

In the pupil position detection step, the processor may acquire the area of the dark region in the pupil search area by performing a binary conversion process that binarizes the luminance values of the pixels in the anterior segment image with reference to the threshold. In this case, pixels having a luminance value equal to or less than the threshold (that is, pixels having a high possibility of being the pupil portion, and hereinafter referred to as “dark pixels”), and pixels having a luminance value greater than the threshold (hereinafter referred to as “bright pixels”) are appropriately divided by the binary conversion process. Moreover, the number of dark pixels included in the pupil search area is proportional to the area of the dark region in the anterior segment image on which the binary conversion process is performed (which may hereinafter be referred to as a “binary-converted image”). Moreover, the number of bright pixels included in the pupil search area is inversely proportional to the area of the dark region. Therefore, a simple process that calculates, from the binary-converted image, for example, the number of dark pixels, the number of bright pixels, a total luminance value (that is proportional to the number of bright pixels), or an average luminance value (that is proportional to the number of bright pixels) in the pupil search area allows acquiring the area of the dark region in the pupil search area appropriately.

However, it is also possible to acquire the area of the dark region in the pupil search area without performing the binary conversion process on the anterior segment image. In this case, for example, the processor may compare the luminance value of each pixel with the threshold, calculate the number of pixels having a luminance value equal to or less than the threshold, and acquire the area of the dark region.

In the pupil position detection step, the processor may further execute a pre-search area setting step. In the pre-search area setting step, the processor sets a pre-search area smaller than the pupil search area in a part of the image area of the anterior segment image, and acquires the area of the dark region in the set pre-search area. In the pupil search area setting step, the processor sets the pupil search area centered on the pre-search area in the image area of the anterior segment image if the area of the dark region in the pre-search area is greater than a reference value. In this case, the processor can omit the process of setting the pupil search area and acquiring the area of the dark region if the area of the dark region in the pre-search area is equal to or less than the reference value. Moreover, the pre-search area is smaller than the pupil search area; accordingly, the burden of processing related to the pre-search area is smaller than the burden of processing related to the pupil search area. Hence, the processor performs the processing related to the pre-search area and accordingly can detect the position of the pupil with high accuracy while preventing an increase in the burden of processing.

The processor may acquire the area of the dark region in the pre-search area by use of the above-mentioned binary-converted image. In this case, the burden of processing on the processor is further reduced as compared to the case where the luminance value of each pixel is compared with the threshold.

The ophthalmic apparatus may further include an axial target projecting optical system configured to emit target light along an optical axis that coincides with the examination axis and projects a target on the examination axis. The shape of the pre-search area may be a two-dimensional shape that surrounds a blank at the center. When the position of the ophthalmic apparatus relative to the examinee's eye is adjusted to an appropriate position, the target that is projected by the axial target projecting optical system becomes a raster on the cornea (for example, the corneal apex when alignment is complicated) of the examinee's eye and appears in the anterior segment image. The luminance value of the raster portion is high. Therefore, even if the set pre-search area appropriately coincides with the pupil center and the corneal apex, when the raster overlaps both of the pupil and the pre-search area in the anterior segment image, the area of the dark region in the pre-search area is reduced. As a result, the position of the pupil may not be detected. In contrast, if the shape of the pre-search area is the shape that surrounds the blank at the center, then the raster is located in the blank at the center of the pre-search area and therefore the pre-search area overlaps the area around the raster. Hence, the position of the pupil is detected with high accuracy even if the raster is projected on the examination axis.

A specific shape of the pre-search area can be set as appropriate. The pre-search area may have, for example, a ring shape (such as an annular shape or polygonal ring shape). The ring shape may be a continuous ring shape, or a ring shape formed discontinuously. Moreover, the pre-search area may not have a ring shape but, for example, a U-shape.

The shape of the pupil search area may be a two-dimensional shape that surrounds a blank at the center. In this case, the accuracy to acquire the area of the dark region in the pupil search area, in addition to the accuracy to acquire the area of the dark region in the pre-search area, is improved. Therefore, the accuracy to detect the position of the pupil is further improved. Various shapes (such as a ring shape and a U-shape) can be adopted also as a specific shape of the pupil search area as in the shape of the pre-search area. Moreover, the pupil search area is larger than the pre-search area. Accordingly, even if the raster overlaps a part of the pupil search area, influence on the accuracy to detect the position of the pupil may not be large. In such a case, the pupil search area may be formed in a shape without a blank at the center.

Moreover, if the center of at least one of the pre-search area and the pupil search area is designed to be blank, the blank is desirably larger in size than the raster formed on the cornea. Moreover, the size of the blank is desirably set in such a manner that the raster fits in the blank even if the alignment of the apparatus relative to the examinee's eye in a direction along the examination axis is insufficient, and the raster formed on the cornea is increased in size.

The ophthalmic apparatus may further include a driver. The driver moves the positions of the anterior segment imaging optical system and the examination-purpose protrusion relative to the examinee's eye. The driver may further execute a position adjustment step of controlling the drive of the driver on the basis of a result of the detection of the position of the pupil in the pupil position detection step and automatically adjusting the positions of the anterior segment imaging optical system and the examination-purpose protrusion relative to the examinee's eye. In this case, the position of the apparatus relative to the examinee's eye is adjusted (that is, aligned) automatically at an appropriate level on the basis of the position of the pupil of the examinee's eye that is detected with high accuracy.

The method for detecting the position of the pupil of an examinee's eye while the influence of the shadow of the examination-purpose protrusion that appears in an anterior segment image is removed is not limited to the method that uses a result of the acquisition of the area of a dark region in a pupil search area. For example, if the positional relationship between the anterior segment imaging optical system and the examination-purpose protrusion is fixed, an area where the shadow of the examination-purpose protrusion overlaps in the image area of an anterior segment image to be captured is located in a fixed position. Therefore, the processor may detect the position of the pupil on the basis of position information on the area where the shadow of the examination-purpose protrusion overlaps in the anterior segment image. For example, if two areas including a dark region whose area is equal to or greater than a reference area are detected from the anterior segment image, then the processor may detect one of the two detected areas, one that is different from the area where the shadow of the examination-purpose protrusion overlaps, as an area including the position of the pupil.

### Embodiment

One of typical embodiments according to the present disclosure is described below with reference to the drawings. An ophthalmic apparatus 1 examines the eye (examinee's eye) E of an examinee while an examination axis IO coincides with the examinee's eye E. The ophthalmic apparatus 1 illustrated by example in the embodiment includes an examination-purpose protrusion (a nozzle in the embodiment) 9 that protrudes toward the examinee's eye along the examination axis IO. The examination-purpose protrusion 9 blows fluid on the cornea of the examinee's eye E to measure the pressure inside the examinee's eye E on the basis of the deformed shape of the cornea. In other words, the ophthalmic apparatus 1 illustrated by example in the embodiment is a non-contact tonometer. However, the ophthalmic apparatus to which the technology illustrated by example in the present disclosure can be applied is not limited to a non-contact tonometer. At least a part of the technology illustrated by example in the present disclosure can be applied to, for example, various ophthalmic apparatuses including the examination-purpose protrusion (such as an ophthalmologic photographing apparatus including an attachment that increases the angle of view, as the examination-purpose protrusion, and an ophthalmic apparatus including an examination-purpose protrusion that emits light for an examination). Moreover, at least a part of the technology illustrated by example in the present disclosure (for example, a technology for detecting the position of the pupil by use of a pre-search area 91 and a pupil search area 92, the technology being described below) may be applied to an ophthalmic apparatus without the examination-purpose protrusion. Examples of the ophthalmic apparatus to which the technology illustrated by example in the present disclosure can be applied include an eye refractive power measurement apparatus, a corneal curvature measurement apparatus, a fundus camera, an OCT apparatus, and a scanning laser ophthalmoscope (SLO). The term “examination” in the present disclosure includes the measurement and photographing of the examinee's eye E.

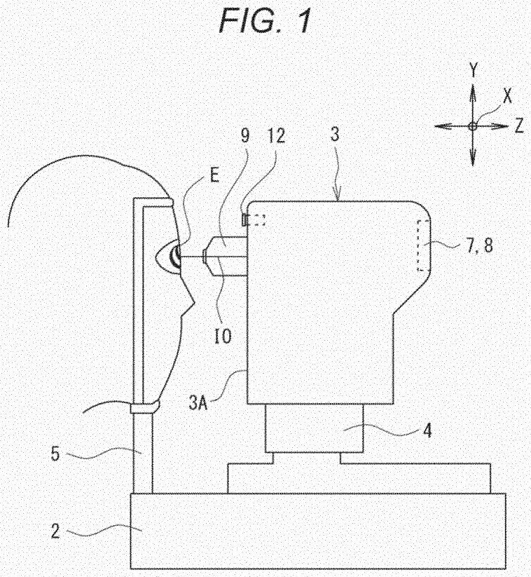

A schematic configuration of the ophthalmic apparatus 1 is described with reference to FIG. 1. In the following description, assume that in FIG. 1 the right-left direction on the page is a Z direction (the front-back direction), the up-down direction on the page is a Y direction (the up-down direction), and the direction into and out of the page is an X direction (the right-left direction). Specifically, assume that in FIG. 1 the left side of the page (the examinee's side) is the front side of the ophthalmic apparatus 1, and the right side of the page is the back side of the ophthalmic apparatus 1. Assume that in FIG. 1 the upper side of the page is the upper side of the ophthalmic apparatus 1, and the lower side of the page is the lower side of the ophthalmic apparatus 1. Assume that in FIG. 1 the side out of the page is the left side of the ophthalmic apparatus 1, and the side into the page is rightward relative to the ophthalmic apparatus 1.

As illustrated in FIG. 1, the ophthalmic apparatus 1 according to the embodiment includes a base 2, a housing 3, a driver 4, and a face support 5. The base 2 is placed in an installation location thereof, and supports the entire ophthalmic apparatus 1. The housing 3 includes various configurations for executing an examination on the examinee's eye E (the details are described below). The housing 3 is supported by the base 2 via the driver 4. The face support 5 supports the face of the examinee and determines the position of the face. In the embodiment, a chin rest and a forehead rest are used as the face support 5. The examinee places the chin on the chin rest and the forehead on the forehead rest, which determines the position of the face. The driver 4 moves the position of the housing 3 relative to the examinee's face that is positioned by the face support 5.

As an example, the driver 4 according to the embodiment causes an actuator such as a motor to move the housing 3 relative to the base 2 in the front-back direction, the up-down direction, and the right-left direction (three-dimensional directions) and accordingly moves the position of the housing 3 relative to the examinee's face (or the examinee's eye). However, it is also possible to modify the configuration of the driver. For example, the driver may move the face support 5 to move the position of the housing 3 relative to the examinee's face. Moreover, the driver may move the housing 3 and the face support 5 together. For example, the driver may move the housing 3 in the front-back direction and in the right-left direction, and move the face support 5 in the up-down direction to move the position of the housing 3 relative to the examinee's face.

The housing 3 includes the examination-purpose protrusion (nozzle) 9, a face imaging optical system 12, a display 7, and an operating device 8. The examination-purpose protrusion 9 protrudes along the examination axis IO toward the examinee's eye from an examinee's eye facing surface 3A being a surface of the housing 3 on a side where the examinee's face is positioned (the front side that faces the examinee's eye in the embodiment). The examination axis IO is aligned with the examinee's eye E when an examination is executed. As an example, the examination-purpose protrusion 9 according to the embodiment is a nozzle through which fluid (for example, compressed air) is blown on the cornea of the examinee's eye E. However, a specific configuration of the examination-purpose protrusion can be selected as appropriate according to, for example, the type of examination that the ophthalmic apparatus executes. For example, an attachment that is detachably mounted on the housing 3 to switch the angle of view of a photographing image, or a protrusion that emits, for example, light or ultrasound waves for an examination from an end thereof to the examinee's eye E, may be used as the examination-purpose protrusion.

The face imaging optical system 12 photographs the face of the examinee. The display 7 displays various images. In the embodiment, the display 7 is placed on the back side of the housing 3 that faces an examiner. Various operation instructions of a user are inputted into the operating device 8. As an example, a touchscreen placed on a display surface of the display 7 is used as the operating device 8 in the embodiment. However, at least any of, for example, a joystick, a mouse, a keyboard, a trackball, a button, and a remote controller may be used as the operating device 8.

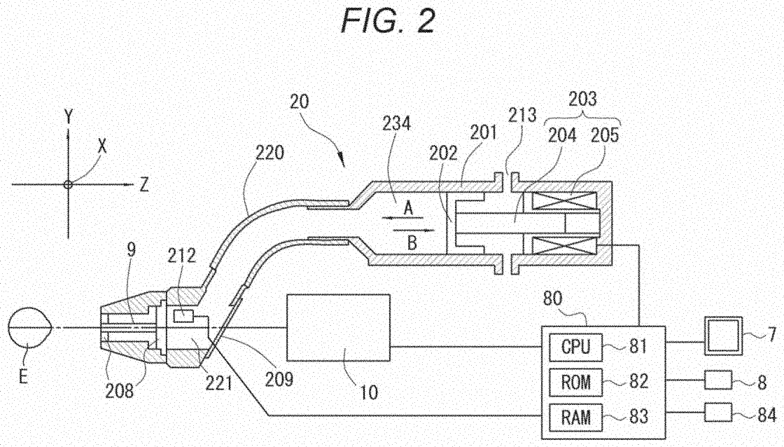

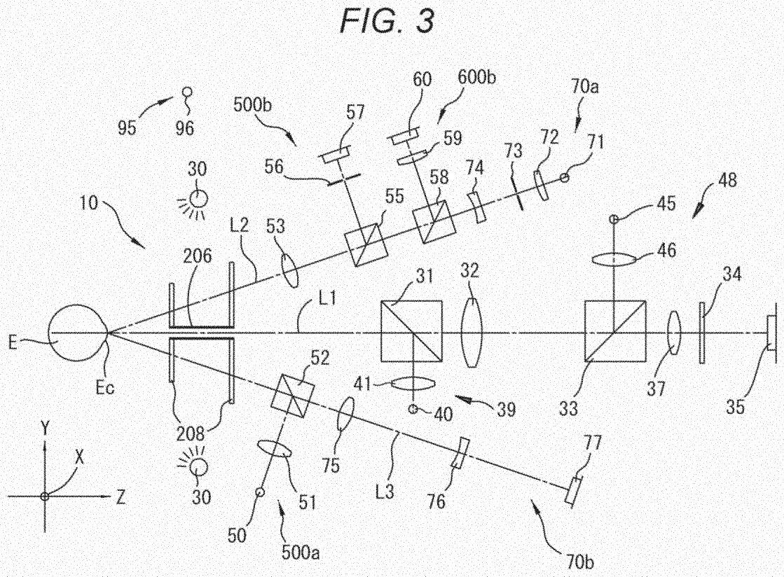

An internal configuration of the ophthalmic apparatus 1 is described with reference to FIG. 2. The ophthalmic apparatus 1 includes a measuring optical system 10, a fluid blower 20, and a control device 80. The measuring optical system 10 and the fluid blower 20 are examples of an examination system that executes an examination on the examinee's eye. As described above, the examination system according to the embodiment measures the pressure inside the examinee's eye in a non-contact manner. The details of the measuring optical system are described below with reference to FIG. 3.

The fluid blower 20 blows fluid on the cornea of the examinee's eye E. The fluid blower 20 includes, for example, a cylinder 201, a piston 202, a solenoid actuator (which may hereinafter be referred to as the solenoid) 203, and the examination-purpose protrusion 9. The cylinder 201 and the piston 202 are used as an air compression mechanism that compresses air to be blown into the examinee's eye. The cylinder 201 has, for example, a cylindrical shape. The piston 202 slides along the axial direction of the cylinder 201. The piston 202 compresses air in an air compression chamber 234 in the cylinder 201. The solenoid 203 includes a movable body 204 and a coil 205. For example, a magnetic substance such as a permanent magnet is used as the movable body 204. When electric current flows through the coil 205, a magnetic field is generated inside the coil 205. The movable body 204 is moved in a direction A in FIG. 2 by an electromagnetic force carried by the magnetic field. The movable body 204 is fixed to the piston 202 with, for example, an unillustrated screw, bolt, and nut. Therefore, the piston 202 travels together with the movable body 204. The movable body 204 travels to move the piston 202 in a compression direction (or a forward direction of travel, the direction A in FIG. 1). The examination-purpose protrusion 9 blows the compressed air to the outside of the apparatus.

The fluid that is compressed by the travel of the piston 202 in the air compression chamber 234 in the cylinder 201 is blown from the examination-purpose protrusion 9 to the cornea of the examinee's eye E through a tube (or may be a pipe) 220 coupled to the end of the cylinder 201, and an airtight chamber 221 that accommodates the compressed air.

Moreover, the solenoid 203 according to the embodiment can change the direction of travel of the movable body 204 by changing the direction of current flowing through the coil 205. For example, when forward current is flown through the coil 205, the movable body 204 travels in the compression direction (the forward direction of travel, the direction A in FIG. 2). When reverse current is flown through the coil 205, the movable body 204 travels in the opposite direction (a reverse direction of travel, a direction B in FIG. 2). The ophthalmic apparatus 1 moves the piston 202 in the direction A, compresses the fluid in the air compression chamber 234, and then moves the piston 202 in the direction B and accordingly can return the piston 202 to the initial position.

The fluid blower 20 includes a glass plate 208 and a glass plate 209. The glass plate 208 is transparent, and holds the examination-purpose protrusion 9 and transmits observation light and target light. The glass plate 209 forms a back wall of the airtight chamber 221, and transmits the observation light and the target light.

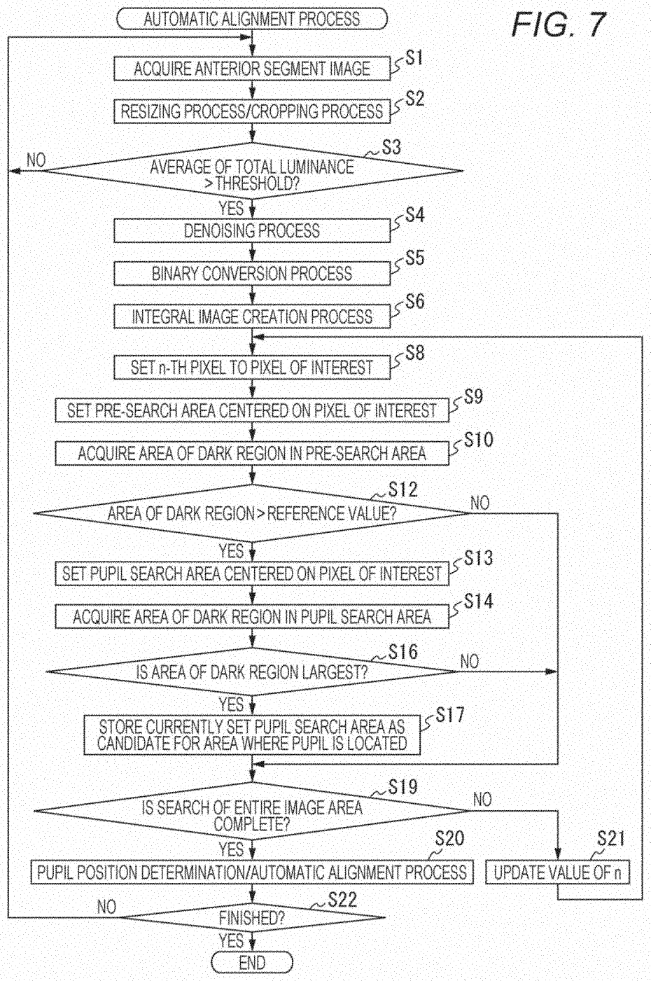

The control device 80 includes a CPU (controller) 81, a ROM 82, and a RAM 83. The CPU 81 is responsible for various types of control over the ophthalmic apparatus 1. For example, various programs and initial values are stored in the ROM 82. Various kinds of information are temporarily stored in the RAM 83. The control device 80 is connected to the display 7, the operating device 8, and a storage 84. The storage (for example, non-volatile memory) 84 is a non-transitory storage medium that can hold stored contents even if power is shut off. For example, a hard disk drive, flash memory, or a detachable USB flash drive may be used as the storage 84. In the embodiment, for example, an ophthalmic apparatus control program for executing an automatic alignment process (refer to FIG. 7) described below is stored in the storage 84. The control device 80 is further connected to, for example, the driver 4, the measuring optical system 10, and the face imaging optical system 12

The optical systems of the ophthalmic apparatus 1 are described with reference to FIG. 3. The ophthalmic apparatus 1 includes an infrared illumination light source 30 that illuminates the examinee's eye. The infrared illumination light source 30 may serve also as at least a part of a target light projecting system that projects a target to the examinee's eye E. An image of the anterior segment of the examinee's eye that is illuminated by the infrared illumination light source 30 is formed on an anterior segment imaging optical system (for example, a CCD camera) 35 via a beam splitter 31, an objective lens 32, a dichroic mirror 33, an imaging lens 37, and a filter 34 (the above configuration may be referred to as the anterior segment photographing optical system). A photographing optical axis L1 of the anterior segment imaging optical system 35 coincides with the examination axis TO (refer to FIG. 1). Therefore, the photographing optical axis L1 of the anterior segment imaging optical system 35 reaches the examinee's eye through the examination-purpose protrusion 9 (refer to FIGS. 1 and 2). Hence, the shadow of the examination-purpose protrusion 9 tends to appear in an image of the anterior segment of the examinee's eye captured by the anterior segment imaging optical system 35. The filter 34 has characteristics that transmits the light of the light source 30 and the light of an infrared light source 40 for alignment and is impermeable to the light of a light source 50 for detecting the deformation of the cornea, which is described below, and visible light. The image formed on the anterior segment imaging optical system 35 is displayed on the display 7.

The light source 40 is a part of an axial target projecting optical system 39 that projects a target to the examinee's eye E at a position through which the examination axis IO passes (that is, on the examination axis TO). The axial target projecting optical system 39 emits target light along the optical axis L1 that coincides with the examination axis IO to project a target being a raster to the cornea (the corneal apex when in alignment) of the examinee's eye E. The axial target projecting optical system 39 includes a projection lens 41 and the beam splitter 31. Infrared light that is projected from the light source 40 through the projection lens 41 is reflected by the beam splitter 31, and projected to the examinee's eye E from the front. The target formed on the cornea (the cornea raster) by the light source 40 forms an image on the anterior segment imaging optical system 35 via the beam splitter 31 to the filter 34, and is used to detect alignment in the up-down and right-left directions and evaluate the focus of the anterior segment image.

A fixation optical system 48 includes the optical axis L1, and presents a fixation target to the examinee's eye E from the front. The fixation optical system 48 includes a visible light source (fixation lamp) 45, a projection lens 46, and the dichroic mirror 33, and projects, to the examinee's eye E, light for directing the examinee's eye E to the front. A light source such as an LED or laser is used as the visible light source 45. Visible light emitted from the visible light source 45 passes through the projection lens 46, is reflected by the dichroic mirror 33, passes through the objective lens 32, and then is projected to the fundus of the examinee's eye E. Consequently, the examinee's eye E attains a state of fixating the fixation target in front, and the direction of the visual line is fixed.

A cornea deformation detecting optical system includes a light projecting optical system 500a and a light receiving optical system 500b, and is used to detect the deformed state of a cornea Ec. The optical systems 500a and 500b are placed in the measuring optical system 10 in the examination system, and moved three-dimensionally by the driver 4.

The light projecting optical system 500a includes an optical axis L3 as a light projecting optical axis, and applies illuminating light diagonally to the cornea Ec of the examinee's eye E. The light projecting optical system 500a includes, for example, the infrared light source 50, a collimator lens 51, and a beam splitter 52. The light receiving optical system 500b includes a photodetector 57, and receives reflected light of the illuminating light from the cornea Ec of the examinee's eye E. The light receiving optical system 500b is placed substantially symmetrically about the optical axis L1 with respect to the light projecting optical system 500a. The light receiving optical system 500b includes, for example, a lens 53, a beam splitter 55, a pinhole plate 56, and the photodetector 57, and forms an optical axis L2 as a light receiving optical axis.

The light emitted from the light source 50 is converted into substantially parallel light flux by the collimator lens 51, reflected by the beam splitter 52, and then becomes coaxial (coincides) with the optical axis L3 of a light receiving optical system 70b described below, and is projected to the cornea Ec of the examinee's eye E. The light reflected from the cornea Ec becomes coaxial (coincides) with the optical axis L2 of a light projecting optical system 70a described below, passes through the lens 53, and then is reflected by the beam splitter 55, and received on the photodetector 57 through the pinhole plate 56. The lens 53 is covered with a coating with a characteristic of being impermeable to the light of the light sources 30 and 40. Moreover, the optical system for detecting the deformation of the cornea is placed in such a manner that the amount of light received on the photodetector 57 is maximum when the examinee's eye is in a predetermined deformed state (applanate state).

Moreover, the cornea deformation detecting optical system serves also as a part of a working distance detecting optical system for detecting the working distance (a distance in the Z direction in the embodiment) of the examination system (including the measuring optical system 10 and the examination-purpose protrusion 9) to the examinee's eye E. Specifically, a light projecting optical system of the working distance detecting optical system in the embodiment serves also as the light projecting optical system 500a of the cornea deformation detecting optical system. Moreover, a light receiving optical system 600b of the working distance detecting optical system includes the lens 53, a beam splitter 58, a condenser lens 59, and a position detection device 60.

The illuminating light projected by the light source 50 and reflected from the cornea Ec forms a target image being a virtual image of the light source 50. The light of the target image passes through the lens 53 and the beam splitter 55, and is reflected by the beam splitter 58, passes through the condenser lens 59, and enters the one- or two-dimensional position detection device 60 such as a PSD or line sensor. When the examinee's eye E (the cornea Ec) moves in the working distance direction (the Z direction), the target image of the light source 50 also moves over the position detection device 60. Therefore, the CPU 81 can detect the working distance on the basis of an output signal from the position detection device 60.

A corneal thickness measuring optical system includes the light projecting optical system 70a, the light receiving optical system 70b, and the fixation optical system 48, and is used to measure the corneal thickness of the examinee's eye E. In the embodiment, a part of the light projecting optical system 70a and a part of the cornea deformation detecting optical system and the working distance detecting optical system are shared. The light projecting optical system 70a applies illuminating light (measurement light) diagonally to the cornea Ec of the examinee's eye E. The light projecting optical system 70a includes an illuminating light source 71, a condenser lens 72, a light restricting member 73, a concave lens 74, and the lens 53 that is shared with the cornea deformation detecting optical system. A visible light source or infrared light source (including near-infrared light) is used as the illuminating light source 71. A light source such as an LED or laser is used. The condenser lens 72 condenses the light emitted from the light source 71.

The light restricting member 73 is placed in an optical path of the light projecting optical system 70a, and restricts the light emitted from the light source 71. The light restricting member 73 is placed in a position that is substantially conjugate to the cornea Ec. For example, a pinhole plate or slit plate is used as the light restricting member 73. The light restricting member 73 is used as an aperture that allows a part of the light emitted from the light source 71 to pass through and blocks the other part of the light. The light projecting optical system 70a forms predetermined pattern light flux (for example, spot light flux or slit light flux) on the cornea of the eye E.

The light receiving optical system 70b includes a photo detector 77, and receives reflected light of the illuminating light from the front and back surfaces of the cornea of the eye E. The light receiving optical system 70b is placed substantially symmetrically about the optical axis L1 with respect to the light projecting optical system 70a. The light receiving optical system 70b includes a light receiving lens 75, a concave lens 76, and the photo detector 77, and forms the optical axis L3 as the light receiving optical axis.

The light emitted from the illuminating light source 71 is condensed by the condenser lens 72, and illuminates the light restricting member 73 from behind. The light from the light source 71 is restricted by the light restricting member 73, and then forms an image (is condensed) near the cornea Ec by means of the lens 53. For example, a pinhole image (if a pinhole plate is used), or a slit image (if a slit plate is used) is formed near the cornea Ec. At this point in time, the light from the light source 71 forms an image near an intersection with the visual axis on the cornea Ec. The reflected light of the illuminating light from the cornea Ec travels in a direction that is symmetric about the optical axis L1 with respect to the projection light flux. The light receiving lens 75 then forms an image of the reflected light on a light receiving surface on the photo detector 77.

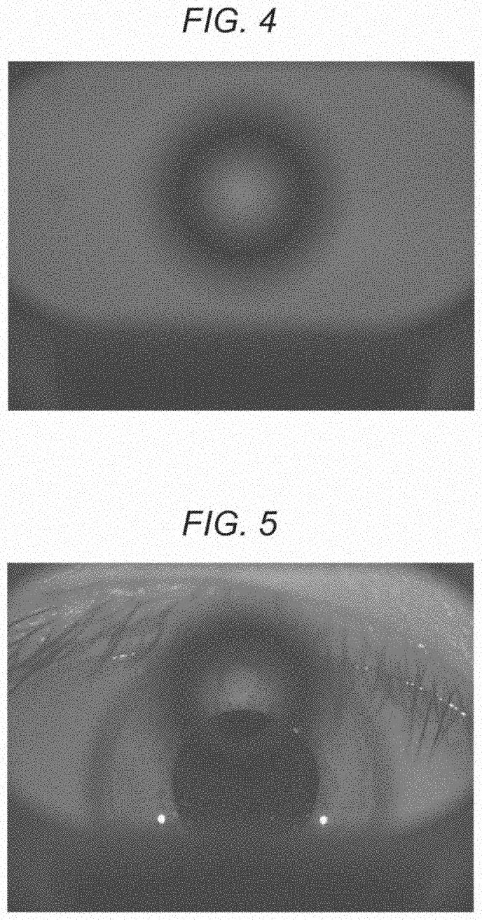

Features of an image captured by the anterior segment imaging optical system 35 of the ophthalmic apparatus 1 according to the embodiment are described with reference to FIGS. 4 to 6. All of FIGS. 4 to 6 are images captured by the anterior segment imaging optical system 35 of the same ophthalmic apparatus 1. The examination axis IO (refer to FIG. 1) of the ophthalmic apparatus 1 passes slightly above the centers of the images illustrated in FIGS. 4 to 6.

FIG. 4 is an image of not the examinee's eye E but the skin of the examinee captured by the anterior segment imaging optical system 35 for reference. In the image illustrated in FIG. 4, there is a ring-shaped dark area slightly above the center of the image although the skin of the examinee was photographed. The dark area is an area centered on the examination axis IO (refer to FIG. 1). The dark area is the shadow of the examination-purpose protrusion 9 protruding toward the photographing target (the skin in FIG. 4). In terms of the structure of the ophthalmic apparatus 1, it is difficult to prevent the appearance of the shadow illustrated by example in FIG. 4.

FIG. 5 is an anterior segment image that was captured by the anterior segment imaging optical system 35 while the pupil center of the examinee's eye E was located below the examination axis IO. As illustrated in FIG. 5, the pupil of the examinee's eye.

E appears darker in a portion where the shadow of the examination-purpose protrusion 9 does not appear than tissues around the pupil (the iris, the sclera, and the eyelid sequentially from the position nearest to the pupil in the area that appears in the image of FIG. 5). However, even the brightness of the tissues around the pupil decreases due to the influence of the shadow in a portion where the shadow of the examination-purpose protrusion 9 overlaps. As a result, for example, the boundary between the pupil and the iris are significantly unclear due to the influence of the shadow. Therefore, if the method for detecting the boundary between the pupil and the iris is adopted as the method for detecting the position of the pupil, then detection accuracy decreases. Moreover, an area where brightness is generally reduced due to the shadow of the examination-purpose protrusion 9 may be incorrectly detected as the pupil region also in the method for detecting an area having a low total or average luminance value of a plurality of pixels, as an area where the pupil is located.

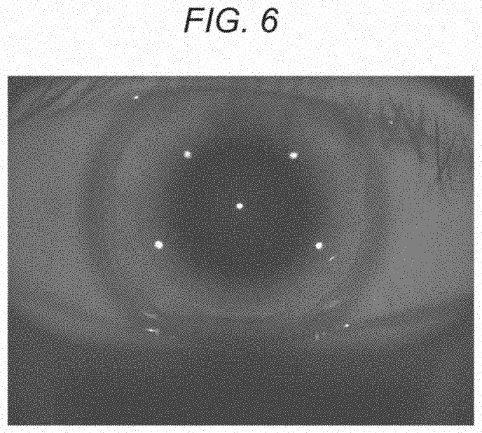

FIG. 6 is an anterior segment image that was captured by the anterior segment imaging optical system 35 while the examination axis IO coincided with the corneal apex and the pupil center of the examinee's eye E (that is, in situations where the alignment of the ophthalmic apparatus 1 with the examinee's eye E was completed). In the state illustrated in FIG. 6, not only the alignment related to the X and Y directions but also the alignment related to the Z direction (that is, the direction along the examination axis IO) was completed. As illustrated in FIG. 6, the shadow of the examination-purpose protrusion 9 overlaps the boundary between the pupil and the iris in the anterior segment image captured in situations where the alignment was completed. As a result, the entire boundary between the pupil and the iris is unclear. Therefore, if the method for detecting the boundary between the pupil and the iris is adopted as the method for detecting the position of the pupil, then the detection accuracy decreases even in situations where the alignment is completed.

Moreover, as illustrated in FIG. 6, the target projected on the examination axis IO by the axial target projecting optical system 39 may appear as a raster on the cornea in the anterior segment image captured by the ophthalmic apparatus 1 according to the embodiment. The raster appears on the corneal apex of the examinee's eye especially in situations where the alignment is completed. Therefore, it is desirable to be able to detect the position of the pupil with high accuracy even if a raster appears on the cornea.

An example of the automatic alignment process that is executed by the ophthalmic apparatus 1 according to the embodiment is described with reference to FIGS. 7 to 9. In the automatic alignment process, the position of the examination system (such as the measuring optical system 10) relative to the examinee's eye E is adjusted automatically. In the embodiment, when the position of the examination system relative to the examinee's eye E is adjusted automatically at an appropriate level by the automatic alignment process, an examination for the examinee's eye E by the examination system is executed automatically. When the user inputs an instruction to execute an automatic alignment (an automatic examination in the embodiment), the CPU 81 of the ophthalmic apparatus 1 executes the automatic alignment process illustrated by example in FIG. 7 in accordance with the ophthalmic apparatus control program stored in the storage 84.

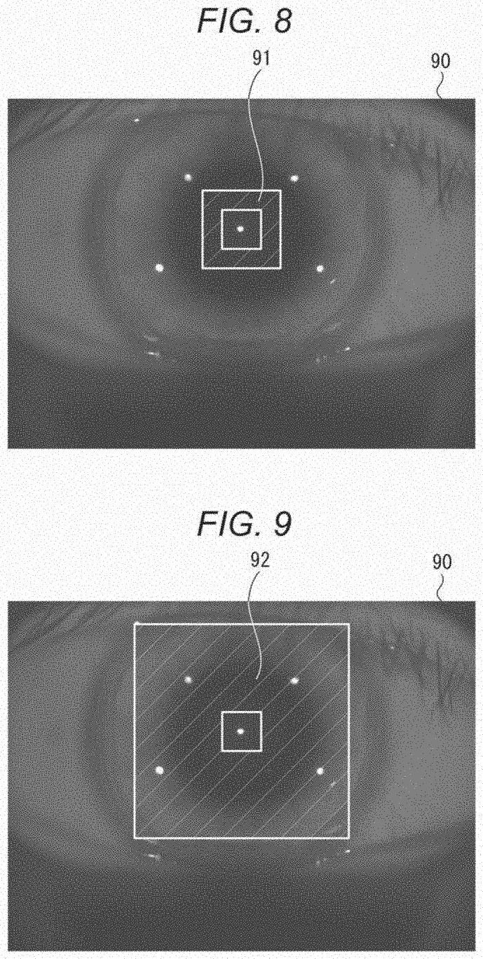

Firstly, the CPU 81 acquires an anterior segment image 90 (refer to FIGS. 8 and 9) of the examinee's eye E captured by the anterior segment imaging optical system 35 (S1). The anterior segment imaging optical system 35 intermittently captures anterior segment images. In S1 according to the embodiment, the latest anterior segment image is acquired from a plurality of images captured continuously.

Next, the CPU 81 executes a resizing process and a cropping process on the anterior segment image acquired in S1 (S2). In the resizing process, the size (resolution) of the anterior segment image acquired in S1 is reduced to encourage an increase in the speed of processing to be executed later on the anterior segment image. As an example, the size (resolution) of the image is reduced to a quarter in the embodiment. Moreover, in the cropping process, an area that is highly likely to be unnecessary to detect the position of the pupil is removed from the anterior segment image acquired in S1. The area to be removed is an outer area of the anterior segment image, an area having a high possibility that the shadows of, for example, the eyelid, the eyelashes, and various members (such as the airtight chamber 221 illustrated in FIG. 2) appear. As a result of the cropping process, an increase in the speed of processing to be executed later on the anterior segment image is encouraged. It is also possible to omit at least one of the resizing process and the cropping process.

Next, the CPU 81 judges whether or not the average of the luminance of the entire anterior segment image acquired in S1 and S2 is greater than a threshold (S3). The luminance value of the entire image to be captured is lower if there are no photographing targets such as the eye and skin of the examinee within the photographing area of the anterior segment imaging optical system 35 than if there are photographing targets. Therefore, if the average luminance of the entire anterior segment image acquired in S1 and S2 is equal to or less than the threshold (S3: NO), it is highly likely that there are no photographing targets within the photographing area of the anterior segment imaging optical system 35. Consequently, the procedure returns to S1 as it is. If the average luminance of the entire image is greater than the threshold (S3: YES), the procedure moves on to S4. A process for detecting the position of the pupil is performed. In the process of S3, instead of the average of the luminance of the entire anterior segment image, the total of the luminance values of a plurality of pixels forming the image may be compared with a threshold.

When starting the process for detecting the position of the pupil, the CPU 81 executes a denoising process on the anterior segment image acquired in S1 and S2 first (S4). A specific method of the denoising process can be selected as appropriate. Noise in the anterior segment image may be removed by, for example, a Gaussian filter, an averaging filter, a median filter, a bilateral filter, or a low-pass filter.

Next, the CPU 81 executes a binary conversion process that binarizes the luminance values of the pixels in the anterior segment image with reference to a threshold (S5). The pixels in the anterior segment image on which the binary conversion process is performed (hereinafter referred to as a “binary-converted image”) include pixels having a luminance value equal to or less than the threshold (hereinafter referred to as “dark pixels”), and pixels having a luminance value greater than the threshold (hereinafter referred to as “bright pixels”). Therefore, the dark pixels having a high possibility of being a pupil portion and the bright pixels having a high possibility of being a portion other than the pupil are appropriately divided according to the threshold in the binary-converted image. Moreover, in an arbitrary area in the binary-converted image, the number of dark pixels is proportional to the area of a region occupied by the dark pixels (hereinafter referred to as a “dark region”), and the number of bright pixels is inversely proportional to the area of the dark region. Hence, in processes of S10 and S14 (described in detail below) to be executed later, the CPU 81 can appropriately acquire the area of a dark region in an arbitrary area simply by calculating, from a binary-converted image, for example, the number of dark pixels, the number of bright pixels, a total luminance value (that is proportional to the number of bright pixels), or an average luminance value (that is proportional to the number of bright pixels) in the arbitrary area.

As illustrated in FIG. 5, in many cases, the luminance of a portion of the tissues around the pupil that appear in the anterior segment image, a portion where the shadow of the examination-purpose protrusion 9 overlaps, is lower than the luminance of a portion where the shadow does not overlap, but is higher than the luminance of the pupil portion. Therefore, the threshold for comparison with a luminance value (in the embodiment, the threshold that is used when the binary conversion process is performed) is set at an appropriate value (in the embodiment, a threshold for dividing the luminance value of the pupil portion and the luminance value of the tissue around the pupil where the shadow of the examination-purpose protrusion 9 overlaps) to appropriately divide the dark pixels having a high possibility of being the pupil portion and the bright pixels having a high possibility of being a portion other than the pupil.

In the embodiment, the CPU 81 sets the threshold for comparison with a luminance value in response to an instruction inputted via the operating device 8. Therefore, the user can specify an appropriate threshold in accordance with various circumstances (such as the brightness of a location where the ophthalmic apparatus 1 is installed). However, it is also possible to change the method for setting the threshold. For example, the CPU 81 may acquire information on the luminance value of the tissue around the pupil where the shadow of the examination-purpose protrusion 9 overlaps in the anterior segment image captured, and set a threshold between the acquired luminance value and the luminance value of the pupil portion. Moreover, the threshold may be a fixed value.

Next, the CPU 81 creates an integral image obtained by integrating the luminance values from the binary-converted image (S6). The integral image is created to increase the speed of various processes to be executed later on the image.

Next, the CPU 81 sets, as a pixel of interest, the n-th pixel (the initial value of n is “1”) of a plurality of pixels in grid form in the anterior segment image (specifically, the anterior segment image on which, for example, the denoising process and the binary conversion process is performed, in the embodiment) (S8). The CPU 81 sets a pre-search area centered on the set pixel of interest in the anterior segment image (S9).

FIG. 8 is an explanatory diagram for explaining an example of the state where the pre-search area 91 is set in the anterior segment image 90. For convenience of description, in order to facilitate understanding of the description, the anterior segment image before the above-mentioned resizing process and binary conversion process are performed is used as the anterior segment image 90 illustrated in FIGS. 8 and 9. The pre-search area 91 is set to previously judge in a simplified manner whether or not a region around the pixel of interest is the dark region having the possibility of being the pupil before a pupil position detection process (S13 to S17) described below is executed. Therefore, the pre-search area 91 is smaller than the pupil search area 92 (refer to FIG. 9) that is set in the pupil position detection process (S13 to S17) described below.

Next, the CPU 81 acquires the area of the dark region in the pre-search area 91 set in the anterior segment image 90 (S10). As described above, in S10 according to the embodiment, for example, the number of dark pixels, the number of bright pixels, the total luminance value, or the average luminance value in the pre-search area 91 is calculated from the binary-converted image to appropriately acquire the area of the dark region in the pre-search area 91. If the area of the dark region in the pre-search area 91 is large, the pre-search area 91 is highly likely to be set in a position where the pupil appears, as illustrated in FIG. 8. On the other hand, if the area of the dark region in the pre-search area 91 is small, the pre-search area 91 is highly likely to be set in a position other than the pupil.

In the anterior segment image 90 illustrated in FIG. 8, a target projected by the axial target projecting optical system 39 (refer to FIG. 3) appears as a raster on the cornea (the corneal apex in FIG. 8). The luminance value of the raster portion is high. Therefore, even when the pre-search area 91 set on the anterior segment image 90 coincides with the position where the pupil appears, if the raster overlaps both of the pupil and the pre-search area 91 in the anterior segment image 90, the area of the dark region in the pre-search area 91 is small. As a result, even if the pre-search area 91 actually coincides with the position of the pupil, it may be judged that the pre-search area 91 does not coincide with the position of the pupil.

Therefore, as illustrated in FIG. 8, the shape of the pre-search area 91 according to the embodiment is formed in a two-dimensional shape that surrounds a blank at the center. As a result, the raster is located in the blank at the center of the pre-search area 91; consequently, the pre-search area 91 overlaps an area around the raster. Hence, the position of the pupil is detected with high accuracy even if the raster is projected on the examination axis IO.

The shape of the pre-search area 91 according to the embodiment is a continuous rectangular ring shape. However, the shape of the pre-search area may be changed to, for example, an annular shape or U-shape. Moreover, the blank at the center of the pre-search area 91 is desirably larger in size than the raster formed on the cornea. In this case, the possibility that the raster overlaps the pre-search area 91 when the raster is located at the center of the blank of the set pre-search area 91 decreases further. Moreover, the outer shape of the pre-search area 91 may be a size that fits in the pupil of the examinee's eye E.

The description returns to FIG. 7. The CPU 81 judges whether or not the area of the dark region in the pre-search area 91 is greater than a reference value (S12). As described above, if the area of the dark region in the pre-search area 91 is equal to or less than the reference value (S12: NO), the pre-search area 91 is highly likely to be set in a position other than the pupil. In this case, even if the pupil position detection process (S13 to S17) described below is executed on the pupil search area 92 (refer to FIG. 9) centered on the pixel of interest, the possibility of detecting the position of the pupil is low. Therefore, the procedure moves on to S19 as it is.

If the area of the dark region in the pre-search area 91 is greater than the reference value (S12: YES), the pupil search area 92 (refer to FIG. 9) centered on the pixel of interest, which was set in S8, is set in the image area of the anterior segment image 90 (S13). In other words, in S13, the pupil search area 92 that is centered on the pre-search area 91 set in S9 and is larger than the pre-search area 91 is set in the anterior segment image 90.

Next, the CPU 81 acquires the area of the dark region in the pupil search area 92 set in the anterior segment image 90 (S14). As described above, in S14 according to the embodiment, for example, the number of dark pixels, the number of bright pixels, the total luminance value, or the average luminance value in the pupil search area 92 is calculated from the binary-converted image to appropriately acquire the area of the dark region in the pupil search area 92.

As illustrated in FIG. 9, the shape of the pupil search area 92 according to the embodiment is formed in a two-dimensional shape that surrounds a blank at the center as in the shape of the pre-search area 91. Hence, even if the raster is projected on the examination axis IO, the area of the dark region in the pupil search area 92 is acquired with high accuracy. Various shapes (such as a ring shape and a U-shape) can be adopted as a specific shape of the pupil search area 92 as in the shape of the pre-search area 91. The blank at the center of the pupil search area 92 is desirably larger in size than the raster formed on the cornea. In the embodiment, the blank at the center of the pupil search area 92 coincides with the blank at the center of the pre-search area 91. Moreover, the outer shape of the pupil search area 92 may have a size that can cover the entire pupil of the examinee's eye E.

Next, the CPU 81 judges whether or not the area of the dark region acquired this time in the process of S14 is the largest among the areas of the dark regions that were acquired repeatedly in S14 while changing the set position of the pupil search area 92 in one anterior segment image 90 acquired in S1 (S16). The pupil is highly likely to be located in the pupil search area 92 including a dark region of the largest area. If the area of the dark region acquired in S14 is not the largest (S16: NO), the procedure moves on to S19 as it is. If the area of the dark region acquired in S14 is the largest (S16: YES), the currently set pupil search area 92 is stored as a candidate for the area where the pupil is located (S17).

Next, whether or not a search of the entire anterior segment image acquired in S1 for the position of the pupil is completed is judged (S19). If the search is not complicated (S19: NO), the value of n for setting a pixel of interest is updated (S21). The procedure returns to S8. The processes of S8 to S17 are repeated. When the search of the entire anterior segment image for the position of the pupil is completed (S19: YES), the pupil search area 92 that was stored in S17 as the candidate for the area where the pupil is located is determined as the area where the pupil is located (S20). Moreover, the CPU 81 controls the drive of the driver 4 (refer to FIG. 1) on the basis of the determined (detected) position of the pupil to automatically adjust the positions of the anterior segment imaging optical system 35 and the examination-purpose protrusion 9 relative to the examinee's eye E (S20). Next, if the automatic alignment is continued (S22: NO), the procedure returns to S1. The pupil position detection process is then executed on the latest anterior segment image. If the automatic alignment is finished (for example, if the examination for the examinee's eye E is finished), the automatic alignment process is ended.

The technology disclosed in the above embodiment is a mere example. Therefore, it is also possible to modify the technology illustrated by example in the above embodiment. For example, the pupil detection process that uses the pre-search area 91 and the pupil search area 92 is executed on the basis of the area of a dark region in each area in the above embodiment. As a result, the position of the pupil is detected with high accuracy regardless of the influence of the shadow of the examination-purpose protrusion 9. However, for example, if the influence of the shadow of the examination-purpose protrusion 9 is small, the pupil detection process that uses the pre-search area 91 and the pupil search area 92 may be executed on the basis of not the area of a dark region in each area but the total or average of luminance values in each area.

In the above embodiment, the blank is provided at the center of each of the pre-search area 91 and the pupil search area 92 to suppress the influence of a raster that appears on the cornea. However, for example, if the influence of a raster that appears on the cornea is small, the blank may not be provided at the center of each of the pre-search area 91 and the pupil search area 92.

In the above embodiment, preprocessing that uses the pre-search area 91 is adopted to reduce the burden of processing that uses the pupil search area 92 larger than the pre-search area 91. However, it is also possible for the ophthalmic apparatus 1 to detect the position of the pupil by use of the pupil search area 92 alone without using the pre-search area 91.

The process of acquiring an anterior segment image in S1 in FIG. 7 is an example of the “anterior segment image acquisition step.” The process of detecting the position of the pupil in S4 to S21 in FIG. 7 is an example of the “pupil position detection step.” The process of setting the pupil search area 92 in S13 in FIG. 7 is an example of the “pupil search area setting step.” The process of acquiring the area of the dark region in S14 in FIG. 7 is an example of the “dark region's area acquisition step.” The process of detecting the pupil search area 92 including a dark region whose area is the largest in S6 and S17 in FIG. 7 is an example of the “area detection step.” The process of setting the pre-search area 91 in S9 in FIG. 7 is an example of the “pre-search area setting step.”

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. An ophthalmic apparatus that examines an examinee's eye while an examination axis coincides with the examinee's eye, the ophthalmic apparatus comprising:
a housing:
an examination-purpose protrusion protruding along the examination axis toward the examinee's eye from an examinee's eye facing surface being a surface of the housing that faces the examinee's eye;
an anterior segment imaging optical system configured to capture an image of the anterior segment of the examinee's eye; and
a processor, wherein
the processor executes:
an anterior segment image acquisition step of acquiring the anterior segment image captured by the anterior segment imaging optical system; and
a pupil position detection step of processing the acquired anterior segment image and detecting the position of the pupil of the examinee's eye while the influence of the shadow of the examination-purpose protrusion that appears in the anterior segment image is removed.

2. The ophthalmic apparatus according to claim 1, wherein in the pupil position detection step, the processor
sets a pupil search area in a part of the image area of the anterior segment image,
acquires the area of a dark region being a region having a luminance value equal to or less than a threshold in the set pupil search area, and
detects the position of the pupil of the examinee's eye on the basis of the acquired area of the dark region.

3. The ophthalmic apparatus according to claim 2, wherein in the pupil position detection step, the processor performs a binary conversion process that binarizes the luminance values of pixels in the anterior segment image with reference to the threshold and acquires the area of the dark region in the pupil search area.

4. The ophthalmic apparatus according to claim 2, wherein in the pupil position detection step, the processor
sets a pre-search area smaller than the pupil search area in a part of the image area of the anterior segment image,
acquires the area of the dark region in the set pre-search area, and
upon the area of the dark region in the pre-search area being greater than a reference value, sets the pupil search area centered on the pre-search area in the image area of the anterior segment image.

5. The ophthalmic apparatus according to claim 4, further comprising an axial target projecting optical system configured to emit target light along an optical axis that coincides with the examination axis and project a target on the examination axis, wherein the shape of the pre-search area is formed in a two-dimensional shape that surrounds a blank at the center.

6. The ophthalmic apparatus according to claim 5, wherein the shape of the pupil-search area is formed in a two-dimensional shape that surrounds a blank at the center.

7. A recording medium where an ophthalmic apparatus control program to be executed by an ophthalmic apparatus that examines an examinee's eye while an examination axis coincides with the examinee's eye is recorded, wherein
the ophthalmic apparatus includes:
a housing:
an examination-purpose protrusion protruding along the examination axis toward the examinee's eye from an examinee's eye facing surface being a surface of the housing that faces the examinee's eye;
an anterior segment imaging optical system configured to capture an image of the anterior segment of the examinee's eye; and
a processor, and
the ophthalmic apparatus control program is executed by the processor of the ophthalmic apparatus to cause the ophthalmic apparatus to execute:
an anterior segment image acquisition step of acquiring the anterior segment image captured by the anterior segment imaging optical system; and
a pupil position detection step of processing the acquired anterior segment image and detecting the position of the pupil of the examinee's eye while the influence of the shadow of the examination-purpose protrusion that appears in the anterior segment image is removed.
